(19) United States
(12) Patent Application Publication
(10) Pub. No.: US 2023/0413809 A1
(43) Pub. Date: Dec. 28, 2023

(54) DISINFECTANT COMPOSITION FOR SURFACES AND THE PREPARATION THEREOF

(71) Applicants: Prerna GORADIA, MAHARASHTRA (IN); Onyx Lotus, LLC, Dover, DE (US)

(72) Inventor: Prerna GORADIA, Maharashtra (IN)

(21) Appl. No.: 18/252,018

(22) PCT Filed: Nov. 5, 2021
(86) PCT No.: PCT/US2021/058366

§ 371 Date: May 5, 2023

(30) Foreign Application Priority Data
Nov. 5, 2020 (IN) 202021048286
Nov. 5, 2020 (IN) 202021048287
Nov. 5, 2020 (IN) 202021048288

(51) Int. Cl. A01N 25/12; A01N 25/30; A01N 25/04; A01N 59/16; A01N 25/34; A01P 1/00

(52) U.S. Cl. CPC A01N 25/12; A01N 25/30; A01N 25/04; A01N 59/16; A01N 25/34; A01P 1/00; B29C 41/14

(57) ABSTRACT

Disinfectant compositions having long-term stability and efficacy are disclosed. More specifically, the compositions of the present disclosure are stable when subjected to both light and heat and efficacious against virus families including the SARS-CoV2 virus. The compositions comprise a nanoparticle anti-microbial agent, and one or more additional components selected from reducing agents, surface binding agents, wetting agents, matrix components, polymers, and reverse micellar surfactants. Methods for treating or impregnating textiles, including gloves, with the disinfectant composition comprising a nanoparticle anti-microbial agent are also disclosed.

### CROSS REFERENCE TO RELATED APPLICATIONS

This application claims priority to Indian Patent Application No. 202021048288 filed Nov. 5, 2020, Indian Patent Application No. 202021048287 filed Nov. 5, 2020; and Indian Patent Application No. 202021048286 filed Nov. 5, 2020, the contents of which are hereby incorporated by reference in their entireties.

### FIELD OF THE INVENTION

The present invention relates to a disinfectant composition and its preparation, as well as its use. More specifically, the disinfectant compositions of the present invention comprise nano-sized anti-microbial agents, which exhibits long-term stability and efficacy against several virus families including the SARS-CoV2 virus.

### BACKGROUND OF THE INVENTION

Currently, no effective drug exists to treat SARS-CoV2 infection. The urgency of the outbreak has led to millions of people being affected globally. Intensive efforts are under way to gain more insight into the mechanisms of viral replication, in order to develop targeted antiviral therapies and vaccines. However, development of medicines and vaccines may take few years.

Various alcohol based disinfectants have been launched which are more effective against bacteria. These products are available in solution, gel and spray form for use on human hand and body surfaces as well as on non-human surfaces such as wood, textile, metal, polymer surface etc. However, their efficacy against viruses has not been established.

Accordingly, it is desired to develop an efficient and stable formulation, which enables the effective treatment of surfaces with anti-viral compound, and which will effectively prevent the spread of the virus.

### SUMMARY OF THE INVENTION

One aspect of the present disclosure encompasses a disinfectant composition comprising a nano-sized anti-microbial agent, which composition can be applied on or impregnated in textiles and various surfaces including plastic, rubber, cardboard, wood, latex, and metal.

Another aspect of the present disclosure encompasses a disinfectant composition which is effective against different viruses, including the SARS-CoV2 virus, and also several strains of bacteria and molds.

Another aspect of the present disclosure encompasses a method for the preparation of the disinfectant composition.

In an additional aspect, the present disclosure encompasses a disinfectant composition, which is stable to light and heat.

In yet another aspect, the present disclosure encompasses a method for the preparation of rubber or nitrile gloves coated and/or impregnated with a disinfectant composition comprising nanoparticles.

### DETAILED DESCRIPTION OF THE INVENTION

Before describing several exemplary embodiments of the invention, it is to be understood that the invention is not limited to the details of construction or process steps set forth in the following description. The invention is capable of other embodiments and of being practiced or being carried out in various ways.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments. The articles “a” and “an” are used herein to refer to one or to more than one (i.e., to at least one) of the grammatical object of the article.

In the present disclosure, “%” refers to “wt. %” or “mass %”, unless otherwise stated.

The present disclosure encompasses a disinfectant composition containing nano-sized anti-microbial agent, and methods of making and using the composition.

### I. COMPOSITION

One aspect of the present disclosure encompasses a disinfectant composition comprising a nanoparticle anti-microbial agent.

A. Active Nanoparticle Anti-Microbial Agent

In accordance with one embodiment, the active nanoparticle anti-microbial agent of the disinfectant nanoparticle composition comprises a metal compound. Silver is currently approved for use in numerous medical applications. It is a naturally occurring ‘green’ material that has been used for thousands of years and is known for its antimicrobial properties. However, the controlled synthesis of stable silver nanoparticles (AgNP's) that do not undergo surface oxidation at the same time in order to provide controlled release of Ag⁺ ions continues to be a major challenge and which is addressed through this invention.

In accordance with one embodiment, the active nanoparticle anti-microbial agent of the disinfectant composition comprises a silver compound. In another embodiment, the active nanoparticle anti-microbial agent of the disinfectant composition comprises a combination of silver compound and copper compound.

The amount of the active nanoparticle anti-microbial agent in the disinfectant composition can and will vary depending on the active agent, the composition, among other variables, and can be determined experimentally. When the active anti-microbial agent is a silver compound, the amount of silver compound in the disinfectant composition may range from between about 0.1 wt % to about 10 wt %. In various aspects, the amount of silver compound in the disinfectant composition may range from about 0.5 wt % to about 5 wt %. In certain aspects, the amount of silver compound in the disinfectant composition may be about 0.5, 0.6, 0.7, 0.8. 0.9, 1, 1.5, 2, 2.5, 3, 3.5, 4, 4.5, or about 5 wt % of the composition.

B. Other Components

The disinfectant compositions disclosed herein comprise one or more additional components selected from the groups consisting of polymers, surfactants, reducing agent(s), complexing agent(s), wetting agent(s), surface binder(s), vehicles, other additives and combinations thereof.

Typically, the polymer is selected from the group consisting of Polyacrylamide, poly(acrylamide-co-acrylic acid), Poly(vinyl alcohol) (PVA), poly vinyl pyrrolidone (PVP), carboxy methyl cellulose (CMC) and polyethylene oxide (PEO) and combinations thereof. Typically, the amount of polymer in the disinfectant composition may range between about 0.1 to about 20 wt %. In one embodiment, the amount of polymer in the disinfectant composition may range between about 1.0 to about 5.0 wt %. In certain aspects, the amount of polymer in the disinfectant composition may be about 1, 1.5, 2, 2.5, 3, 3.5, 4, 4.5, or about 5 wt % of the composition.

The surfactants may be selected from the group consisting of Kolliphor EL, Poloxamer 407, Tween 80, sodium lauryl sulfate, sodium carboxy methyl cellulose, calcium carboxy methyl cellulose, hydrogenated or non-hydrogenated glycerolipids, ethoxylated or non-ethoxylated, linear or branched, saturated or mono- or polyunsaturated C₆ to C₃₀ fatty acids in the form of the acid or an alkali metal or its salt, cyclodextrin, sodium lauryl sulfate, alkaline earth metal or amine salt ethoxylated or non-ethoxylated esters of sucrose, sorbitol, sorbitan monooleate, mannitol, glycerol or polyglycerol containing from 2 to 20 glycerol units, or glycol with said fatty acids, mono-, di- or triglycerides or mixtures of glycerides of said fatty acids, ethoxylated or non-ethoxylated, linear or branched, saturated or mono- or polyunsaturated C₆ to C₃₀ fatty alcohols, cholesterol and derivatives thereof, other derivatives with a sterol skeleton, ethoxylated or non-ethoxylated ethers of sucrose, sorbitol, mannitol, glycerol or polyglycerol containing from 2 to 20 glycerol units, or glycol with said fatty alcohols, hydrogenated or non-hydrogenated, polyethoxylated vegetable oils, polyoxyethylene/polyoxypropylene block polymers (poloxamers), polyethylene glycol hydroxystearate, sphingolipids and sphingosine derivatives, polyalkyl glucosides, ceramides, polyethylene glycol/alkyl glycol copolymers, and polyethylene glycol/polyalkylene glycol ether di-block or tri-block copolymers, Triton X, diacetylated monoglycerides, diethylene glycol monostearate, ethylene glycol monostearate, glyceryl monooleate, glyceryl monostearate, propylene glycol monostearate, macrogol stearate 400, macrogol stearate 2000, polyoxyethylene 50 stearate, macrogol ethers, cetomacrogol 1000, lauramacrogols, nonoxinols, octoxinols, tyloxapol, poloxamers, polysorbate 20, polysorbate 40, polysorbate 60, polysorbate 65, polysorbate 80, polysorbate 85, sorbitan monolaurate, sorbitan monooleate, sorbitan monopalmitate, sorbitan monostearate, sorbitan sesquioleate, sorbitan trioleate, sorbitan tristearate and sucrose esters.

Additionally or alternatively, the surfactant may be a reverse micellar surfactant selected from the group of strong ionic detergents such as but not limited to cetyltrimethylammonium bromide or chloride, sodium salt of cholic acid, sodium salt of deoxycholic acid, sodium salt of dioctyl sulfosuccinate, sodium salt of dodecyl sulphate and sodium salt of N-lauroylsarcosine and sodium lauryl ether sulphate (SLES). Ionic detergents contain a head group which is either positively or negatively charged. For example, the anionic detergent sodium dodecyl sulfate (SDS) carries a negatively charged sulfate group on a linear C12 hydrocarbon chain. SDS is considered to be a very strong and biologically harsh surfactant and is able to denature proteins by breaking intra- and intermolecular interactions, thus destroying their biological activity. Other anionic detergents like the bile acid salts Na-cholate and Na-deoxycholate have a rigid steroidal core structure. They do not carry a well-defined polar head group opposite to a hydrophobic tail (such as SDS). The polar groups are distributed on different parts of the molecule, resulting in a polar and non-polar side, e.g. Na-deoxycholate carries a carboxylate group at the end of a short hydrocarbon chain and two hydroxyl groups on the steroid structure. The bile acids are less denaturing than the ionic alkyl detergents, possibly due to their rigid steroidal ring structure.

Cationic detergents have a positively charged head group, which is often a quaternary ammonium group, e.g. CTAB carries a trimethylammonium group on a C₁₆ hydrocarbon chain. It is also a strong detergent and will often irreversibly denature proteins. The lower the critical micelle concentration of a surfactant, the more is its detergency; for example, the CTAB has a Critical micellar concentration (CMC) of 0.92 mM and the Critical micellar concentration (CMC) of sodium sulfosuccinate is 0.6 mM. Typically, the amount of surfactant present in the disinfectant composition may range between about 0.1 to about 5 wt %. In certain aspects, the amount of surfactant in the disinfectant composition may be about 0.1, 0.2, 0.3, 0.4, 0.5, 0.6, 0.7, 0.8. 0.9, 1, 1.5, 2, 2.5, 3, 3.5, 4, 4.5, or about 5 wt % of the composition.

Non-limiting examples of a matrix component include, but are not limited to, silica, titania, alcohol, or combinations thereof. In one embodiment, titania is added to the disinfectant composition to aid the adsorption of the silver compound in the formulation.

Typically, the reducing agent or complexing agent is selected from the group of organic acids consisting of citric acid, sodium citrate, ascorbic acid and combinations thereof. Additional examples of organic acids include but are not limited to lactic acid, fumaric acid, succinic acid, malic acid, and combinations thereof. Typically, the amount of reducing agent present in the disinfectant composition may be in the range of about 1.0 to about 60 wt %. In one embodiment, the amount of reducing agent present in the disinfectant composition may be in the range of about 10 to about 40 wt %. In certain aspects, the amount of reducing agent in the disinfectant composition may be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 wt % of the composition.

Non-limiting examples of surface binders include, but are not limited to, melamine, adhesives, polymers, acrylates, and the like.

In one embodiment, the disinfectant composition comprises:
a. silver chloride in an amount between about 0.5 to about 5 wt %;
b. at least one polymer in an amount between about 0.1 to about 5.0 wt %; and
c. at least one reverse micellar ionic surfactant in an amount between about 1 to about 10 wt %.

In one embodiment, the disinfectant composition comprises:
a. silver nitrate in an amount between about 0.5 to about 5 wt %;
b. at least one polymer in an amount between about 1.0 to about 5.0 wt %; and
c. at least one reducing agent in an amount between about 10 to about 50 wt %.

In one embodiment, the disinfectant composition comprises:
a. silver nitrate;
b. at least one polymer;
c. at least one reducing agent; and
d. at least one wetting agent.

In one embodiment, the disinfectant composition comprises:
a. silver chloride;
b. at least one polymer;
c. at least one reverse micellar surfactant in an amount between about 1 to about 5 wt % and
d. at least one matrix component.

In one embodiment, the disinfectant composition comprises:
a. silver chloride;
b. at least one polymer;
c. at least one strong ionic detergent such as the dioctyl sodium sulfosuccinate;
d. at least one surface binder such as metal oxide powder; and
e. at least one wetting agent.

In one embodiment, the disinfectant composition comprises:
a. silver nitrate;
b. at least one polymer;
c. at least one reducing agent;
d. at least one surface binder; and
e. at least one wetting agent.

In one exemplary embodiment, the disinfectant composition comprises:
a. silver nitrate; and
b. polyvinyl pyrrolidone.

In one exemplary embodiment, the disinfectant composition comprises:
a. silver nitrate;
b. polyvinyl pyrrolidone;
c. melamine;
d. trisodium citrate; and
e. Triton X.

In one exemplary embodiment, the disinfectant composition comprises:
a. silver chloride in an amount of from about 0.1 to about 4 wt %;
b. polyvinyl pyrrolidone in an amount between about 0.1 to about 2 wt %;
c. alcohol in an amount between about 0.1 to about 5 wt %;
d. sodium docusate in an amount between about 1 to about 10 wt %; and
e. titania in an amount between about 1 to about 10 wt %.

In one exemplary embodiment, the disinfectant composition comprises:
a. silver nitrate;
b. a copper compound;
c. at least one polymer; and
d. at least one reducing agent.

In one embodiment, the composition is in the form of solution.

In one embodiment, the composition is in the form of spray formulation.

In one embodiment, the composition is in the form of a powder or powder spray. In one embodiment, cold spraying or spray drying technique is used for making powder formulation.

### II. METHOD OF APPLYING THE DISINFECTANT COMPOSITION TO VARIOUS MEDIUMS

Another aspect of the present disclosure encompasses the application of the disinfectant composition to various mediums, such as textiles or fabrics. In one embodiment the present disclosure encompasses the application of the disinfectant composition to gloves, including natural rubber latex or nitrile gloves, such as medical examination gloves.

In one embodiment of the present disclosure, the nanoparticle antimicrobial agent is added during the glove formation process to which works by the mechanism of generating a water soluble singlet oxygen generator.

Rubber dipping is one of the stations in the manufacturing process of latex/nitrile or other types of rubber gloves. A tank is filled with compounded latex and a latex layer is formed on a glove former or mold after it moves through this tank. The thickness of the latex on the glove former is determined at the coagulating and dipping stage. The longer the time the glove former travels in the latex tank, the thicker the latex will be on the finished glove product. In one embodiment, the present disclosure encompasses the method of adding the disinfectant composition to the latex tank in a volume percentage of between about 1 to about 10% after the rubber is compounded and several hours before the actual application of the latex to the formers.

In one embodiment, the present disclosure encompasses the method of first contacting a glove former with a coagulant solution comprising divalent calcium cations, carbonate particles, and a water soluble singlet oxygen generator, and then contacting the glove former with a natural rubber latex or nitrile dispersion. In one embodiment, the disinfectant composition in the form of a powder or liquid is mixed with a coagulant solution in the proportion of about 2 to about 10 wt. % and the solution is applied and dried in accordance with regular procedures.

Typically, a method of making nitrile or rubber latex medical examination gloves comprises the steps of: (a) dipping the glove formers into a coagulant solution containing divalent calcium cations and calcium nitrate particles, the coagulant solution further comprising a disinfectant composition comprising nanoparticle anti-microbial agent; (b) dipping the coagulant-coated glove formers into a nitrile or rubber latex dispersion, which dispersion comprises a disinfectant composition comprising nanoparticle anti-microbial agent; and (c) placing the coated nitrile or rubber latex gloves into packaging, wherein the packaging comprises a clear coat varnish, which may be formed of a polyurethane dipping solution, which is impregnated with the disinfectant composition comprising nanoparticle anti-microbial agent.

In another embodiment, there is provided a method of treating various layers of rubber gloves, wherein the method comprises applying the disinfectant composition of the present disclosure to the various layers of the gloves.

In a further embodiment, there is provided an antimicrobial textile or fabric impregnated with the disinfectant composition of the present disclosure. In yet another embodiment, there is provided an antimicrobial material made of polymer or wood or rubber which is impregnated with the disinfectant composition of the present disclosure.

In another embodiment, the present disclosure encompasses a disinfectant composition, which is stable to light and heat. Antibacterial activity of a formulation is typically attributed to electrostatic interactions between cations and the negatively charged membrane surface of microbe and/or to other interactions between the cations and the microbe's RNA/DNA proteins. The formulation of the present invention has a unique mechanism of action: when nanoparticles are in solution, molecules associate with the nanoparticle surface to establish multiple layers of charges that stabilize the particles and prevent aggregation. A specialty polymer is used and the electron potential of the solution is kept in the reductive regime so that the metal remains in a zero valent state. When the composition of the present invention is applied on a surface or impregnated into the gloves, the active nanoparticle ingredient (such as silver) on the surface or glove is a photosensitizer which generates singlet oxygen when exposed to light. The singlet oxygen oxidizes the protein and lipid of the bacteria, and consequently leads to the death of microbes. The reactive ion species that are generated on the surfaces also have antimicrobial action.

In one of the preferred embodiments, silver nanoparticles are suspended in an aqueous citrate buffer, which weakly associates with the nanoparticle surface. This citrate-based agent may be selected because the weakly bound capping agent provides long-term stability and is readily displaced by various other molecules including thiols, amines, polymers, antibodies, and proteins when the release mechanism is triggered.

This special technology coats the metal nanoparticles of controlled size and shape for controlled release (during use) or no release of the ions (during storage). This adaptable design allows one to produce a library of membrane-coated silver nanoparticles (AgNP's) with tuned surface chemistry and optical properties for a variety of surface coating applications. In one example, metal nanoparticle with a protective organic layer based on electrostatic charges is provided.

In a further embodiment, there is provided a process for the preparation of the disinfectant composition of the present disclosure.

In yet another embodiment, there is provided the method of using the disinfectant composition of the present disclosure to treat or disinfect a surface.

In yet another embodiment, there is provided a method of treating a surface, wherein the method comprises applying the disinfectant composition of the present disclosure on the surface. Typically, the step of applying the composition includes spraying, coating, pouring and the like with the disinfectant composition.

In one embodiment, the application step involves techniques such as padding, exhaust and other appropriate methods.

As various changes could be made in the above described compositions and methods without departing from the scope of the invention, it is intended that all matter contained in the above description and in the examples given below, shall be interpreted as illustrative and not in a limiting sense.

### III. EXAMPLES

All patents and publications mentioned in the specification are indicative the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure. Therefore all matter set forth is to be interpreted as illustrative and not in a limiting sense.

### Example 1

### Example 1a

The AgNPs were capped with various hydrophilic polymers such as Polyacrylamide, poly(acrylamide-co-acrylic acid), Poly(vinyl alcohol) (PVA), poly vinyl pyrrolidone and the like which have a tendency to hydrogen bond in aqueous solutions. Other capping agents used could be carboxy methyl cellulose (CMC) and polyethylene oxide (PEO) which are also water-soluble. The specific reducing agent such as ascorbic acid or sodium citrate was used in large proportions such as between about 10 to about 25 wt % of the formulation to yield a specific size and shape of the AgNP which is important for its anti-viral property. The water-soluble polymer was used in about 2 wt % of the formulation. In one example, poly vinyl pyrrolidone K-30 is used. It is a hygroscopic, amorphous polymer that can be plasticized with water and most common plasticizers. It has high polarity and propensity to form bonds with hydrogen donors such as phenols, carboxylic acids, anionic dyes and inorganic salts.

### Example 1b

Starting with the composition from Example 1a, a small amount of polymer such as melamine which a trimer of cyanamide, with a 1,3,5-triazine skeleton was added to give a superior binding ability to fabric. The combination of two different polymer systems provided a unique binding ability of the composition to the surface.

### Example 2

### Example 2a

Silver chloride was capped with various hydrophilic polymers such poly vinyl pyrrolidone which have a tendency to hydrogen bond in aqueous solutions. The water-soluble polymer was used in about 1 wt % of the formulation. The strong ionic detergents mentioned above (not limited to the list above but notably with low CMC values) were used in an amount of up to about 5 wt % of the formulation. Further, titania was added in the weight ratio of about 1 to about 5 wt % of the formulation. A clear powerful disinfectant solution resulted.

### Example 2b

Using the clear powerful disinfectant solution from Example 2a, a small amount of alcohol was added to the formulation, which resulted in a fast drying and homogeneous formulation.

### Example 3

### Example 3a

Silver chloride was capped with various hydrophilic polymers such as poly vinyl pyrrolidone, namely poly vinyl pyrrolidone K-30, which have a tendency to hydrogen bond in aqueous solutions. The water-soluble polymer was used in an amount of about 1 wt % of the formulation. The cationic surfactants mentioned herein were used in an amount of up to about 5 wt % of the formulation. Further silica was added in the weight ratio of about 1 to 5 wt % of the formulation which was then filtered. A clear powerful disinfectant solution resulted.

### Example 3b

To the disinfectant solution of Example 3a, a small amount of a silane, 3-(trimethoxysilyl) propyl dimethyl octadecyl ammonium chloride was added to the formulation to give a superior binding ability to fabric and other surfaces. This combination of cationic quaternary ammonium silanes gives a unique binding ability of the disinfectant composition to the surface.

### Example 4: Preparation of 1 Liter of Liquid Disinfectant Formulation

700 mL water was added to a beaker equipped with a stirrer and a heater assembly. To this, 40 g cetrimonium chloride was added to obtain a mixture which was thoroughly mixed and transparent. To this mixture, 200 mg of AgC1 was then carefully added and mixed well to obtain the second mixture. The order of addition of ingredients is critical as it prevents the oxidation of the silver ions and allows them to remain in the stable and complexed form. To the second mixture, 1 g of polyvinylpyrrolidone (PVP) and 1 g of silica was added and the volume was made up with deionized water. The formulation was then filtered through a 10 micron filter.

### Example 5: Preparation of 1 Liter of Liquid Disinfectant Formulation

700 mL water was added to a flask. 20 grams of polyvinylpyrrolidone (PVP) was added to the water and thoroughly mixed together. 150 g trisodium citrate was added to obtain a mixture which was translucent and clear. To this mixture, 2 grams of AgNO3 was then carefully added and mixed well to obtain a second mixture. The order of addition of ingredients is critical as it prevents the oxidation of the silver ions and allows them to remain in the metallic and complexed form. To the second mixture, 20 grams of polyvinylpyrrolidone (PVP) was added and the volume was made up with deionized water. The formulation was then filtered through a 10 micron filter. This is a key step in the size selection of the nanoparticles. Triton X 100 and up to 3 wt % melamine were then added.

### Example 6

Synthesis of silver reverse micelles with robust hybrid lipid-coated layers was performed. The layers were comprised of ionic detergents such as cetrimonium chloride, cetrimonium bromide or the anionic detergents such as sodium docusate or SLES. This prevented the silver from undergoing surface oxidation and Ag+ ion dissolution. At the same time, the ion was slowly released from the coating after the textile or the surface had been coated.

1. A disinfectant composition comprising:
a. a nanoparticle anti-microbial agent; and
b. at least one polymer

2. The disinfectant composition of claim 1, wherein the nanoparticle antimicrobial agent is a metal compound.

3. The disinfectant composition of claim 2, wherein the metal compound is selected from the group consisting of silver, copper, and combinations thereof.

4. The disinfectant composition of claim 1, wherein the at least one polymer is selected from the group consisting of Polyacrylamide, poly(acrylamide-co-acrylic acid), Poly(vinyl alcohol) (PVA), poly vinyl pyrrolidone (PVP), carboxy methyl cellulose (CMC) and polyethylene oxide (PEO) and combinations thereof.

5. The disinfectant composition of claim 1, wherein the nanoparticle antimicrobial agent is present in the composition in an amount of between about 0.1 wt % to about 10 wt %.

6. The disinfectant composition of claim 1, wherein the nanoparticle antimicrobial agent is present in the composition in an amount of between about 0.5 wt % to about 5 wt %.

7. The disinfectant composition of claim 1, wherein the at least one polymer is present in the composition in an amount of between about 0.1 wt % to about 20 wt %.

8. The disinfectant composition of claim 1, wherein the at least one polymer is present in the composition in an amount of between about 1 wt % to about 5 wt %.

9. The disinfectant composition of claim 1, wherein the composition further comprises a surfactant selected from the group consisting of Kolliphor EL, Poloxamer 407, Tween 80, sodium lauryl sulfate, sodium carboxy methyl cellulose, calcium carboxy methyl cellulose, hydrogenated or non-hydrogenated glycerolipids, ethoxylated or non-ethoxylated, linear or branched, saturated or mono- or polyunsaturated C6 to C30 fatty acids in the form of the acid or an alkali metal or its salt, cyclodextrin, sodium lauryl sulfate, alkaline earth metal or amine salt ethoxylated or non-ethoxylated esters of sucrose, sorbitol, sorbitan monooleate, mannitol, glycerol or polyglycerol containing from 2 to 20 glycerol units, or glycol with said fatty acids, mono-, di- or triglycerides or mixtures of glycerides of said fatty acids, ethoxylated or non-ethoxylated, linear or branched, saturated or mono- or polyunsaturated C6 to C30 fatty alcohols, cholesterol and derivatives thereof, other derivatives with a sterol skeleton, ethoxylated or non-ethoxylated ethers of sucrose, sorbitol, mannitol, glycerol or polyglycerol containing from 2 to 20 glycerol units, or glycol with said fatty alcohols, hydrogenated or non-hydrogenated, polyethoxylated vegetable oils, polyoxyethylene/polyoxypropylene block polymers (poloxamers), polyethylene glycol hydroxystearate, sphingolipids and sphingosine derivatives, polyalkyl glucosides, ceramides, polyethylene glycol/alkyl glycol copolymers, and polyethylene glycol/polyalkylene glycol ether di-block or tri-block copolymers, Triton X, diacetylated monoglycerides, diethylene glycol monostearate, ethylene glycol monostearate, glyceryl monooleate, glyceryl monostearate, propylene glycol monostearate, macrogol stearate 400, macrogol stearate 2000, polyoxyethylene 50 stearate, macrogol ethers, cetomacrogol 1000, lauramacrogols, nonoxinols, octoxinols, tyloxapol, poloxamers, polysorbate 20, polysorbate 40, polysorbate 60, polysorbate 65, polysorbate 80, polysorbate 85, sorbitan monolaurate, sorbitan monooleate, sorbitan monopalmitate, sorbitan monostearate, sorbitan sesquioleate, sorbitan trioleate, sorbitan tristearate and sucrose esters, and combinations thereof.

10. The disinfectant composition of claim 1, wherein the composition further comprises a reverse micellar surfactant selected from the group consisting of cetyltrimethylammonium bromide or chloride, sodium salt of cholic acid, sodium salt of deoxycholic acid, sodium salt of dioctyl sulfosuccinate, sodium salt of dodecyl sulphate and sodium salt of N-lauroylsarcosine and sodium lauryl ether sulphate (SLES), and combinations thereof.

11. The disinfectant composition of claim 1, wherein the composition further comprises at least one reducing agent, at least one surface binder, at least one matrix component, at least wetting agent, or combinations thereof.

12. The disinfectant composition of claim 1, wherein the composition is in the form of a solution.

13. The disinfectant composition of claim 1, wherein the composition is in the form of a powder.

14. A disinfectant composition comprising:
a. silver nitrate; and
b. polyvinyl pyrrolidone.

15. A disinfectant composition comprising:
a. silver chloride in an amount between about 0.5% to about 5 wt %;
b. at least one polymer in an amount between about 0.1% to about 5.0 wt %; and
c. at least one reverse micellar ionic surfactant in an amount between about 1% to about 10 wt %.

16. A disinfectant composition comprising:
a. silver chloride in an amount of from about 0.1% to about 4 wt %;
b. polyvinyl pyrrolidone in an amount between about 0.1% to about 2 wt %;
c. alcohol in an amount between about 0.1% to about 5 wt %;
d. sodium docusate in an amount between about 1% to about 10 wt %; and
e. titania in an amount between about 1% to about 10 wt %.

17. A method of making an anti-microbial nitrile or rubber latex glove, the method comprising the steps of:
a. dipping a glove former into a nitrile or rubber latex dispersion, which dispersion comprises a disinfectant composition comprising a nanoparticle anti-microbial agent to form a coated nitrile or coated rubber latex glove; and
b. placing the coated nitrile or coated rubber latex glove into a packaging, wherein the packaging comprises a clear coat varnish impregnated with a disinfectant composition comprising a nanoparticle anti-microbial agent.

18. A method of making an anti-microbial nitrile or rubber latex glove, the method comprising the steps of:
a. dipping a glove former into a coagulant solution containing divalent calcium cations and calcium nitrate particles, the coagulant solution further comprising a disinfectant composition comprising a nanoparticle anti-microbial agent, to form a coagulant-coated glove former;
b. dipping the coagulant-coated glove former into a nitrile or rubber latex dispersion, which dispersion comprises a disinfectant composition comprising a nanoparticle anti-microbial agent to form a coated nitrile or coated rubber latex glove; and
c. placing the coated nitrile or coated rubber latex glove into a packaging, wherein the packaging comprises a clear coat varnish impregnated with a disinfectant composition comprising a nanoparticle anti-microbial agent.

19. A method of making a stable disinfectant composition, the method comprising the steps of:
a. combining at least one reverse micellar surfactant with water to form a transparent mixture;
b. adding an effective amount of a nanoparticle antimicrobial agent to the transparent mixture to form a second mixture;
c. adding an effective amount of at least one polymer and at least one matrix component to the second mixture to form a resultant formulation; and
d. filtering the resultant formulation to form the stable disinfectant composition.

20. A method of making a stable disinfectant composition, the method comprising the steps of:
a. combining at least one polymer with water to form an initial mixture;
b. adding an effective amount of at least one reducing agent to the initial mixture to form a translucent mixture;
c. adding an effective amount of a nanoparticle antimicrobial agent to the translucent mixture to form a second mixture;
d. adding an effective amount of at least one polymer to the second mixture to form a resultant formulation; and
e. filtering the resultant formulation to form the stable disinfectant composition.
